(19) United States
(12) Patent Application Publication
(10) Pub. No.: US 2025/0175749 A1
(43) Pub. Date: May 29, 2025

(54) SYNCHRONIZED SPECTRAL ANALYSIS

(71) Applicant: Cochlear Limited, Macquarie University, NSW (AU)

(72) Inventors: Michael GOOREVICH, Naremburn, NSW (AU); Hans VANDENWIJNGAERDEN, West Ryde, NSW (AU)

(21) Appl. No.: 18/840,277

(22) PCT Filed: Feb. 21, 2023
(86) PCT No.: PCT/IB2023/051576

§ 371 Date: Aug. 21, 2024

Related U.S. Application Data
(60) Provisional application No. 63/314,674, filed on Feb. 28, 2022.

(51) Int. Cl. H04R 25/00; A61N 1/05; A61N 1/36

(52) U.S. Cl. CPC H04R 25/554; A61N 1/36038; H04R 25/505; A61N 1/0541; H04R 2225/67

(57) ABSTRACT

Presented herein are techniques for synchronized spectral analysis in systems comprising first and second devices.

### BACKGROUND

### Field of the Invention

Aspects of the present invention relate generally to synchronized spectral analysis.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect presented herein, a method is provided. The method comprises: establishing a data link between a first hearing device configured to be disposed at a first side of a head of a user and a second hearing device configured to be disposed at a second side of the head of the user; obtaining a synchronization event from the data link; and using the synchronization event to align spectral analysis of audio signals at the first hearing device with spectral analysis of audio signals at the second hearing device.

In another aspect presented herein, a method is provided. The method comprises: receiving first audio data at a first hearing device of a binaural hearing device system; performing spectral analysis of the first audio data at the first hearing device; aligning a timing of the spectral analysis of the first audio data at the first hearing device with a timing of spectral analysis of second audio data at a second hearing device of the binaural hearing device system; and following the spectral analysis, generating, at the first hearing device, a first sequence of stimulation signals representative of the first audio data.

In another aspect presented herein, a binaural hearing device system is provided. The binaural hearing device system comprises: a first hearing device located at a first ear of a user and including one or more first processors configured to: obtain a first set of audio samples, and capture one or more buffers of the first set of audio samples, a second hearing device located at a second ear of the user and including one or more second processors configured to: obtain a second set of audio samples, and capture one or more buffers of the second set of audio samples, wherein the one or more first processors and the one or more second processors are configured to cooperate to synchronize the capture of the one or more buffers of the first set of audio samples with the capture of the one or more buffers of the second set of audio samples

In another aspect, one or more non-transitory computer readable storage media encoded with instructions are provided. The or more non-transitory computer readable storage media include instructions that, when executed by one or more processors, cause the one or more processors to: establish a data link between the first hearing device and a second hearing device configured to be located at a second side of a head of the user; obtain a synchronization event from the data link; and use the synchronization event to align spectral analysis of audio signals at the first hearing device with spectral analysis of audio signals at the second hearing device.

In another aspect presented herein, a hearing device configured to be worn on a first side of a head of a user is provided. The hearing device comprises: one or more sound inputs configured to receive a first set of sound signals associated with at least one sound source; a wireless transceiver configured to form a data link with a second hearing device configured to be disposed at a second side of the head of the user; and at least one processor configured to perform spectral analysis of the first set of sound signals, wherein a timing of the spectral analysis is based on at least one characteristic of the data link.

In another aspect presented herein, a system is provided. The system comprises: a first sensory device including one or more first processors configured to: obtain a first set of input samples, and capture one or more buffers of the first set of input samples; and a second sensory device including one or more second processors configured to: obtain a second set of input samples, and capture one or more buffers of the second set of input samples, wherein the one or more first processors and the one or more second processors are configured to cooperate to synchronize the capture of the one or more buffers of the first set of input samples with the capture of the one or more buffers of the second set of input samples.

In another aspect, a method is provided. The method comprises: establishing a data link between a first device and a second device; receiving first data at the first device; performing spectral analysis of the first data at the first device; aligning a timing of the spectral analysis of the first data at the first device with a timing of spectral analysis of second data at a second device based on the information obtained from the data link.

### BRIEF DESCRIPTION OF THE DRAWINGS

Embodiments of the present invention are described herein in conjunction with the accompanying drawings, in which:

FIG. 1A is a schematic view of a cochlear implant system in which embodiments presented herein may be implemented;

FIG. 1B is a side view of a recipient wearing the cochlear implant system of FIG. 1A;

FIG. 1C is a schematic view of the components of the cochlear implant system of FIG. 1A;

FIGS. 1D and 1E are block diagrams of sound processing units forming part of the cochlear implant system of FIG. 1A;

FIG. 2A is a schematic diagram illustrating conventional spectral analysis in a binaural hearing device system;

FIG. 2B is a schematic diagram illustrating spectral analysis in a binaural hearing device system, in accordance with certain embodiments presented herein;

FIG. 3 is a schematic diagram illustrating spectral analysis of another hearing device system, in accordance with certain embodiments presented herein;

FIGS. 4A and 4B are schematic diagrams illustrating filter-bank alignment of first and second hearing devices based on a wireless synchronization event, in accordance with embodiments presented herein;

FIG. 5 is a schematic diagram illustrating filter-bank alignment of first and second hearing devices based on a wireless synchronization event with delays, in accordance with embodiments presented herein;

FIG. 6 is a flowchart of a method, in accordance with certain embodiments presented herein;

FIG. 7 is a flowchart of another method, in accordance with certain embodiments presented herein;

FIG. 8 is a schematic diagram illustrating an example system that can be configured to perform synchronized spectral analysis, in accordance with certain embodiments presented herein;

FIG. 9 is a schematic diagram illustrating another example system that can be configured to perform synchronized spectral analysis, in accordance with certain embodiments presented herein; and

FIG. 10 is a schematic diagram illustrating another example system that can be configured to perform synchronized spectral analysis, in accordance with certain embodiments presented herein.

### DETAILED DESCRIPTION

Many devices, such as hearing devices, certain consumer electronics, wearable devices, etc., operate by performing “spectral analysis” of sound signals. As used herein, “spectral analysis” refers to a process to determine the frequency contents of received time domain sound signals (e.g., convert time domain signals into the frequency domain). Presented herein are techniques for synchronized spectral analysis in systems comprising first and second separate/independent devices. That is, the techniques presented herein are configured to enable the first and second devices to perform spectral analysis at the same time on contemporaneous input signals/data.

For ease of illustration, the techniques presented herein are primarily described with reference to hearing device systems comprises of at least two devices that operate to convert sound signals into one or more acoustic, mechanical, and/or electrical stimulation signals for delivery to a user/recipient. The one or more hearing devices that can form part of a hearing device system include, for example, one or more personal sound amplification products (PSAPs), hearing aids, cochlear implants, middle ear stimulators, bone conduction devices, brain stem implants, electro-acoustic cochlear implants or electro-acoustic devices, and other devices providing acoustic, mechanical, and/or electrical stimulation to a recipient.

One specific type of hearing device system, referred to herein as a “binaural hearing device system” or more simply as a “binaural system,” includes two hearing devices, where one of the two hearing prosthesis is positioned at each ear of the recipient. More specifically, in a binaural system each of the two hearing devices provides stimulation to one of the two ears of the recipient (i.e., either the right or the left ear of the recipient). The binaural system can include any combination of one or more personal sound amplification products (PSAPs), hearing aids, middle ear auditory prostheses, bone conduction devices, direct acoustic stimulators, electro-acoustic prostheses, auditory brain stimulators, cochlear implants, combinations or variations thereof, etc., For example, embodiments presented herein can be implemented in binaural systems comprising two hearing aids, two cochlear implants, a hearing aid and a cochlear implant, or any other combination of the above or other devices. As such, in certain embodiments, the techniques presented herein enable synchronized spectral analysis in binaural hearing device systems comprising first and second hearing devices positioned at first and second ears, respectively, of a recipient.

As noted above, it is to be appreciated that the techniques presented herein may be implemented with any of a number of systems, including in conjunction with cochlear implants or other hearing devices, balance prostheses (e.g., vestibular implants), retinal or other visual prostheses, cardiac devices (e.g., implantable pacemakers, defibrillators, etc.), seizure devices, sleep apnea devices, electroporation devices, spinal cord stimulators, deep brain stimulators, motor cortex stimulators, sacral nerve stimulators, pudendal nerve stimulators, vagus/vagal nerve stimulators, trigeminal nerve stimulators, diaphragm (phrenic) pacers, pain relief stimulators, other neural, neuromuscular, or functional stimulators, etc. In further embodiments, the presented herein may also be implemented by, or used in conjunction with, systems comprising remote microphone devices, consumer electronic devices, etc.

Merely for ease of description, aspects of the techniques will be generally described with reference to a specific system, namely a bilateral cochlear implant system. As used herein, a “bilateral cochlear implant system” is a specific type of binaural system that includes first and second cochlear implants located at first and second ears, respectively, of a recipient. In such systems, each of the two cochlear implant system delivers stimulation (current) pulses to one of the two ears of the recipient (i.e., either the right or the left ear of the recipient). In a bilateral cochlear implant system, one or more of the two cochlear implants may also deliver acoustic stimulation to the ears of the recipient (e.g., an electro-acoustic cochlear implant) and/or the two cochlear implants need not be identical with respect to, for example, the number of electrodes used to electrically stimulate the cochlea, the type of stimulation delivered, etc.

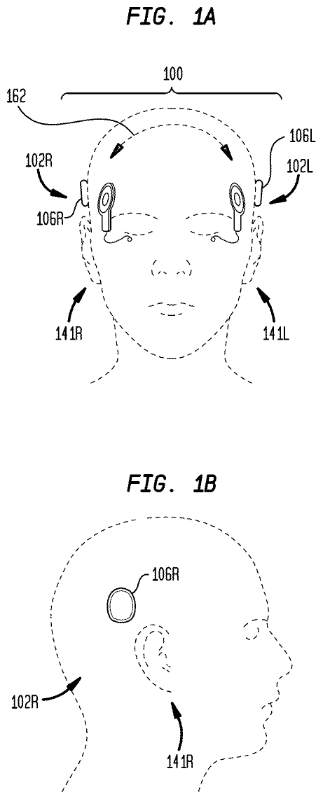

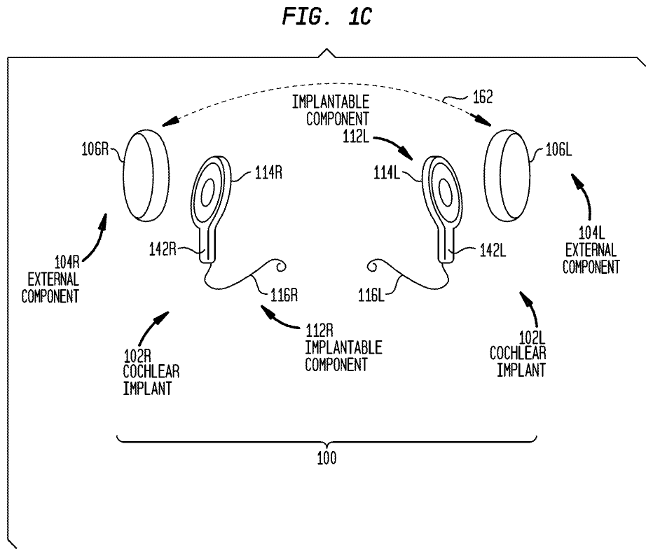

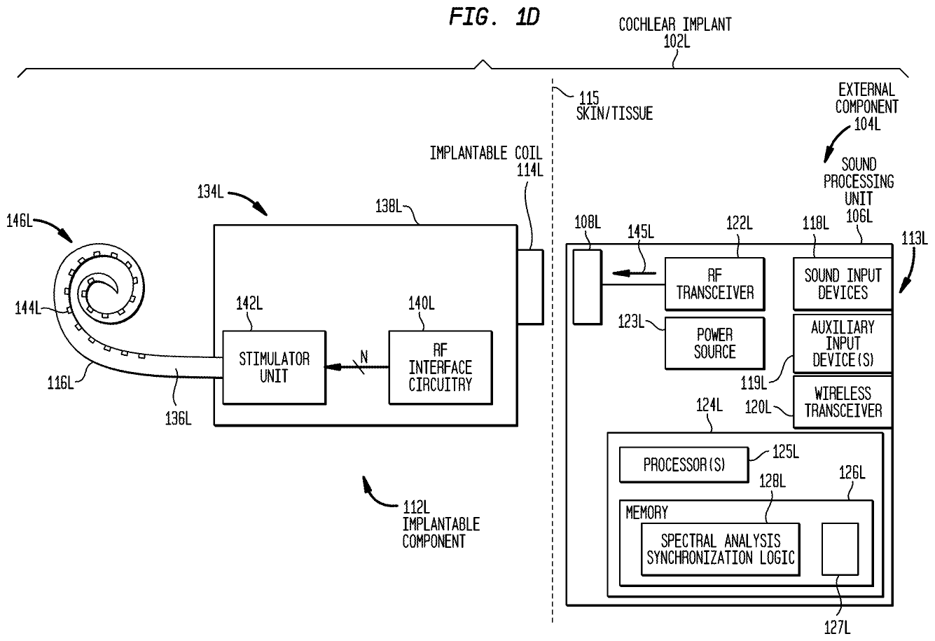

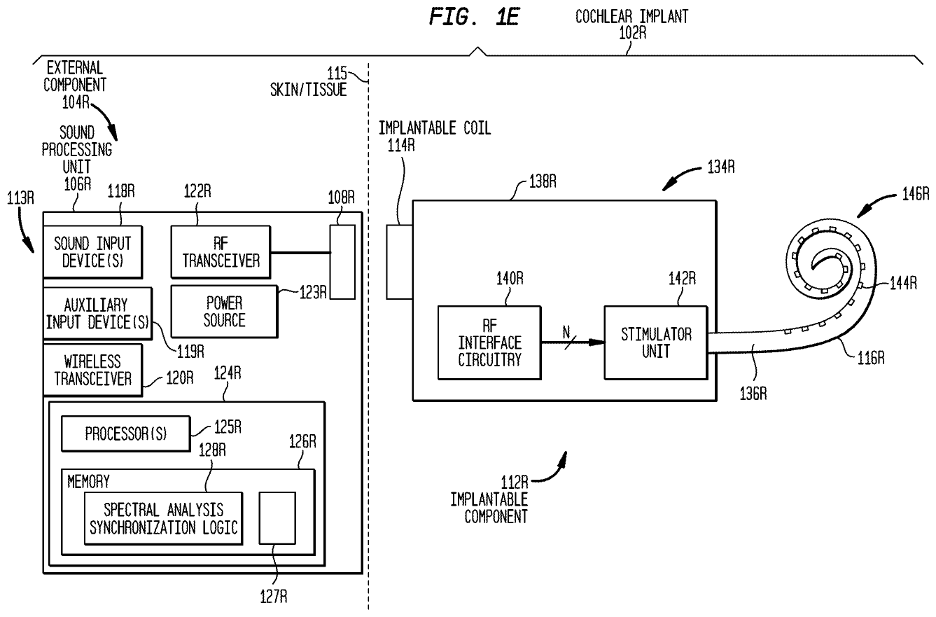

FIGS. 1A-1E are diagrams illustrating one example bilateral cochlear implant system 100 configured to implement the techniques presented herein. More specifically, FIGS. 1A-1E illustrate an example bilateral system 100 comprising left and right cochlear implants, referred to as cochlear implant 102L and cochlear implant 102R. FIGS. 1A and 1B are schematic drawings of a recipient wearing the left cochlear implant 102L at a left ear 141L and the right cochlear implant 102R at a right ear 141R, while FIG. 1C is a schematic view of each of the left and right cochlear implants. FIGS. 1D and 1E are block diagrams illustrating further details of the left cochlear implant 102L and the right cochlear implant 102R, respectively.

Referring specifically to FIG. 1C, cochlear implant 102L includes an external component 104L that is configured to be directly or indirectly attached to the body of the recipient and an implantable component 112L configured to be implanted in the recipient. The external component 104L comprises a sound processing unit 106L, while the implantable component 112L includes an internal coil 114L, a stimulator unit 142L and an elongate stimulating assembly (electrode array) 116L implanted in the recipient's left cochlea (not shown in FIG. 1C).

The cochlear implant 102R is substantially similar to cochlear implant 102L. In particular, cochlear implant 102R includes an external component 104R comprising a sound processing unit 106R, and an implantable component 112R comprising internal coil 114R, stimulator unit 142R, and elongate stimulating assembly 116R.

FIG. 1D is a block diagram illustrating further details of cochlear implant 102L, while FIG. 1E is a block diagram illustrating further details of cochlear implant 102R. As noted, cochlear implant 102R is substantially similar to cochlear implant 102L and includes like elements as that described below with reference to cochlear implant 102L. For ease of description, further details of cochlear implant 102R have been omitted from the description.

As noted, the external component 104L of cochlear implant 102L includes a sound processing unit 106L. The sound processing unit 106L comprises one or more input devices 113L that are configured to receive input signals (e.g., sound or data signals). In the example of FIG. 1D, the one or more input devices 113L include one or more sound input devices 118L (e.g., microphones, audio input ports, telecoils, etc.), one or more auxiliary input devices 119L (e.g., audio ports, such as a Direct Audio Input (DAI), data ports, such as a Universal Serial Bus (USB) port, cable port, etc.), and a wireless transmitter/receiver (transceiver) 120L. However, it is to be appreciated that one or more input devices 113L may include additional types of input devices and/or less input devices (e.g., one or more auxiliary input devices 119L could be omitted).

The sound processing unit 106L also comprises one type of a closely-coupled transmitter/receiver (transceiver) 122L, referred to as or radio-frequency (RF) transceiver 122L, a power source 123L, and a processing module 124L. The processing module 124L comprises one or more processors 125L and a memory 126L that includes sound processing logic 127L and spectral analysis synchronization logic 128L.

In the examples of FIGS. 1A-1E, the sound processing unit 106L and the sound processing unit 106R are off-the-ear (OTE) sound processing units (i.e., components having a generally cylindrical shape and which is configured to be magnetically coupled to the recipient's head), etc. However, it is to be appreciated that embodiments of the present invention may be implemented by sound processing units having other arrangements, such as by a behind-the-ear (BTE) sound processing unit configured to be attached to and worn adjacent to the recipient's ear, including a mini or micro-BTE unit, an in-the-canal unit that is configured to be located in the recipient's ear canal, a body-worn sound processing unit, etc.

The implantable component 112L comprises an implant body (main module) 134L, a lead region 136L, and the intra-cochlear stimulating assembly 116L, all configured to be implanted under the skin/tissue (tissue) 115 of the recipient. The implant body 134L generally comprises a hermetically-sealed housing 138L in which RF interface circuitry 140L and a stimulator unit 142L are disposed. The implant body 134L also includes the internal/implantable coil 114L that is generally external to the housing 138L, but which is connected to the transceiver 140L via a hermetic feedthrough (not shown in FIG. 1D).

As noted, stimulating assembly 116L is configured to be at least partially implanted in the recipient's cochlea. Stimulating assembly 116L includes a plurality of longitudinally spaced intra-cochlear electrical stimulating contacts (electrodes) 144L that collectively form a contact or electrode array 146L for delivery of electrical stimulation (current) to the recipient's cochlea.

Stimulating assembly 116L extends through an opening in the recipient's cochlea (e.g., cochleostomy, the round window, etc.) and has a proximal end connected to stimulator unit 142L via lead region 136L and a hermetic feedthrough (not shown in FIG. 1D). Lead region 136L includes a plurality of conductors (wires) that electrically couple the electrodes 144L to the stimulator unit 142L.

As noted, the cochlear implant 102L includes the external coil 108L and the implantable coil 114L. The coils 108L and 114L are typically wire antenna coils each comprised of multiple turns of electrically insulated single-strand or multi-strand platinum or gold wire. Generally, a magnet is fixed relative to each of the external coil 108L and the implantable coil 114L. The magnets fixed relative to the external coil 108L and the implantable coil 114L facilitate the operational alignment of the external coil 108L with the implantable coil 114L. This operational alignment of the coils enables the external component 104L to transmit data, as well as possibly power, to the implantable component 112L via a closely-coupled wireless link formed between the external coil 108L with the implantable coil 114L. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106L includes the processing module 124L. The processing module 124L is configured to convert received input signals (received at one or more of the input devices 113L) into output signals 145L for use in stimulating a first ear of a recipient (i.e., the processing module 124L is configured to perform sound processing on input signals received at the sound processing unit 106L). Stated differently, in the sound processing mode, the one or more processors 125L are configured to execute sound processing logic stored, for example, in in memory 126L to convert the received input signals into output signals 145L that represent electrical stimulation for delivery to the recipient.

In the embodiment of FIG. 1D, the output signals 145L are provided to the RF transceiver 114, which transcutaneously transfers the output signals 145L (e.g., in an encoded manner) to the implantable component 112L via external coil 108L and implantable coil 114L. That is, the output signals 145L are received at the RF interface circuitry 140L via implantable coil 114L and provided to the stimulator unit 142L. The stimulator unit 142L is configured to utilize the output signals 145L to generate electrical stimulation signals (e.g., current signals) for delivery to the recipient's cochlea via one or more stimulating contacts 144L. In this way, cochlear implant 102L electrically stimulates the recipient's auditory nerve cells, bypassing absent or defective hair cells that normally transduce acoustic vibrations into neural activity, in a manner that causes the recipient to perceive one or more components of the received sound signals.

As noted, cochlear implant 102R is substantially similar to cochlear implant 102L and comprises external component 104R and implantable component 112R. External component 104R includes a sound processing unit 106R that comprises external coil 108R, input devices 113R (i.e., one or more sound input devices 118R, one or more auxiliary input devices 119R, and wireless transceiver 120R), closely-coupled transceiver (RF transceiver) 122R, power source 123R, and processing module 124R. The processing module 124R includes one or more processors 125R and a memory 126R that includes sound processing logic 127R and spectral analysis synchronization logic 128R. The implantable component 112R includes an implant body (main module) 134R, a lead region 136R, and the intra-cochlear stimulating assembly 116R, all configured to be implanted under the skin/tissue (tissue) 115 of the recipient. The implant body 134R generally comprises a hermetically-sealed housing 138R in which RF interface circuitry 140L and a stimulator unit 142L are disposed. The implant body 134R also includes the internal/implantable coil 114R that is generally external to the housing 138R, but which is connected to the RF interface circuitry 140R via a hermetic feedthrough (not shown in FIG. 1E). The stimulating assembly 116R includes a plurality of longitudinally spaced intra-cochlear electrical stimulating contacts (electrodes) 144R that collectively form a contact or electrode array 146R for delivery of electrical stimulation (current) to the recipient's cochlea. Each of the elements of cochlear implant 102R shown in FIG. 1E are similar to like-numbered elements of cochlear implant 102L shown in FIG. 1D.

It is to be appreciated that the arrangements of cochlear implants 102L and 102R, as shown in FIGS. 1A-1E, are merely illustrative and that the cochlear implants 102L and 102R could have different arrangements. For example, in certain embodiments, the implantable components 112L and 112R could each include a wireless transceiver that is similar to the wireless transceivers 120L and 120R. In the same or other embodiments, the implantable components 112L and 112R could each include processing modules that are similar to the processing modules 124L and 124R. The implantable components 112L and 112R could also include processing modules that are not necessarily the same as the processing modules 124L and 124R, for example, in terms of functional capabilities.

The cochlear implants 102L and 102R are configured to establish a binaural wireless communication link/channel 162 (binaural wireless link) that enables the cochlear implants 102L and 102R (e.g., the sound processing units 104L/104R and/or the implantable components 112l/122, if equipped with wireless transceivers) to wirelessly communicate with one another. The binaural wireless link 162 can be, for example, a magnetic induction (MI) link, a standardized wireless channel, such as a Bluetooth®, Bluetooth® Low Energy (BLE) or other channel interface making use of any number of standard wireless streaming protocols, a proprietary protocol for wireless exchange of data, etc. Bluetooth® is a registered trademark owned by the Bluetooth® SIG. The binaural wireless link 162 is enabled by the wireless transceivers 120L and 120R.

The sound processing performed at each of the cochlear implant 102L and the cochlear implant 102R (e.g., at the sound processing units 104L/104R and/or the implantable components 112l/122, if equipped with processing modules) includes some form of spectral analysis (e.g., some process to determine the frequency contents of received time domain sound signals). In certain examples, as described further below, the spectral analysis can include a filter-bank (filterbank) analysis.

The present inventors have recognized that, for a binaural hearing device system, such as bilateral cochlear implant system 100, binaural synchronization of the spectral analysis (e.g., filter-bank analysis window/buffer timing) is important, as the content of each buffer is a factor in the analysis of the input sound that will eventually be converted to stimulation. As such, presented herein are techniques for aligning/synchronizing the spectral analysis across both devices of a binaural hearing device system.

More specifically, referring to the example of FIGS. 1A-1E, the cochlear implant 102L is configured to receive first sound signals (e.g., audio samples), collect subsets/buffers of the first sound signals, and perform spectral analysis on the subsets/buffers of the first sound signals. Similarly, the cochlear implant 102R is configured to receive second sound signals, collect subsets/buffers of the second sound signals, and perform spectral analysis on the subsets/buffers of the second sound signals. The cochlear implants 102L and 102R are configured to align/synchronize the spectral analysis of the subsets/buffers of the first and second sound signals based on information obtained from the binaural wireless link 162 (e.g., based on a synchronization event from establishment of the binaural wireless link). As a result, each iteration of the spectral analysis is performed at the same time, and on contemporaneous audio content, at each of the cochlear implants 102L and 102R. These techniques can be implemented in various different systems having multiple components, such as a systems comprised of two external devices, systems comprised of two implants, systems comprised of two external devices and two implants, systems comprising wireless streamers in combination with external devices and/or implants, etc.

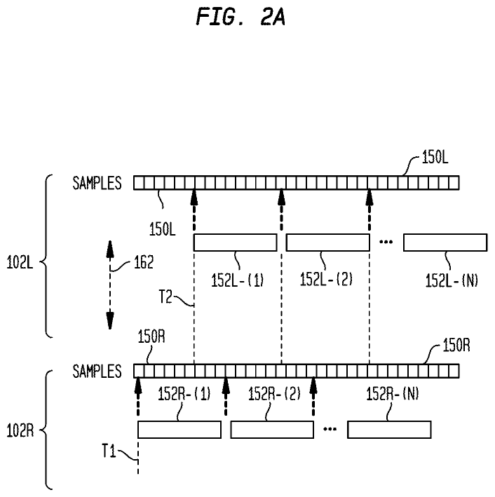

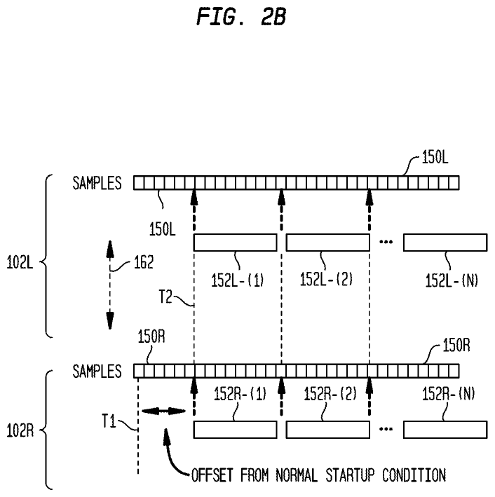

FIG. 2A is a schematic diagram illustrating conventional spectral analysis operations, while FIG. 2B is a schematic diagram illustrating spectral analysis synchronization in accordance with certain embodiments presented herein. For ease of illustration, FIGS. 2A and 2B will be described with reference to cochlear implant system 100 of FIGS. 1A-1E.

As noted above, the cochlear implant 102L and cochlear implant 102R are configured to communicate with one another via a binaural wireless link 162. The cochlear implants 102L/102R establish the binaural wireless link 162 (e.g., over magnetic induction, Bluetooth®, etc.) and implement a synchronization scheme to ensure the link 162 operates to reliably transmit data from one device to the other. That is, the binaural communication channel 162 can operate by defining time slots for communication with a timing synchronization mechanism that the cochlear implants 102L/102R use to prevent collisions of transmitted data. In accordance with embodiments presented herein, the synchronization of the binaural wireless link 162 is used to synchronize the spectral analysis at the cochlear implants 102L and 102R.

For example, in certain embodiments, when established, one or more of the wireless transceivers 120L/120R issues a “synchronization event” indicating that the binaural wireless link 162 is operational and synchronized. This synchronization event indicates a synchronized time base for the binaural wireless link 162. That is, once the synchronization event occurs, each of the wireless transceivers 120L/120R is aware of the precise operational timing of the other. In accordance with embodiments presented, this time base for the binaural wireless link 162 is used to establish a “synchronized spectral analysis time base” at cochlear implants 102L and 102R (e.g., create a “tick” counter or similar scheme at both cochlear implants). In certain embodiments, the synchronized spectral analysis time base can indicate, or be used to determine, a “time offset” or “operational time difference “between the two implants. In certain examples, the synchronized spectral analysis time base is accurate to a closest audio sample at the audio sample rate of the cochlear implants 102L and 102R.

In accordance with embodiments presented herein, the synchronized spectral analysis time base is used at the processing module 124L (e.g., by the spectral analysis synchronization logic 128L) and the processing module 124R (e.g., by the spectral analysis synchronization logic 128R) for binaural synchronization of the spectral analysis operations. More specifically, as shown in FIGS. 2A and 2B, each of the cochlear implants 102L and 102R obtains/generates a plurality of audio samples representing, for example, audio signals received via the respective sound input elements. The audio samples obtained at the cochlear implant 102L are referred to as audio samples 150L, while the audio samples obtained at the cochlear implant 102R are referred to as audio samples 150R.

As noted above, “spectral analysis” refers to a process to determine the frequency contents of received time domain sound signals (e.g., convert time domain signals into the frequency domain). In hearing devices, such as cochlear implants 102L and 102R, spectral analysis is performed repeatedly, where each spectral analysis run/iteration converts a different group/subset (buffer) of audio samples into the frequency domain. That is, a spectral analysis is performed on only a subset of the received audio samples collected during a previous period of time, for example, on audio samples collected in a buffer. For example, audio sampling is performed at a certain rate, such as at a rate of 20 kilohertz (kHz) (i.e., 20,000 audio samples a second), while spectral analysis is performed at a lower rate, such as at a rate of 1 kHz (i.e., 1,000 times a second). This means that, in this illustrative example, a new spectral analysis is started for each 20 audio samples that are received.

As noted above, the embodiments presented herein are used to ensure binaural alignment of the spectral analysis at each of the cochlear implants 102L and 102R. That is, the techniques presented herein enable the cochlear implant 102L and cochlear implant 102R to perform spectral analysis at the same time, meaning the resulting frequency domain signals will correspond to “contemporaneous audio content.” As used herein, the “contemporaneous audio content” refers to audio samples that represent/correspond to sounds captured/received during the same period of time (e.g., capture audio samples at each of the cochlear implants 102L and 102R that represent sounds received during the same time period by the corresponding sound input elements).

For example, cochlear implant 102L collects different buffers 152L-(1)-152L-(N) of the audio samples 150L for spectral analysis, while cochlear implant 102R collects different buffers 152R-(1)-152R-(N) of the audio samples 150R for spectral analysis. As shown in FIG. 2A, if the cochlear implants 102L and 102R were to operate independently (e.g., without the benefit of the techniques presented herein), then cochlear implant 102R would initiate capture of the buffers 152R at a first time point (T1) and the cochlear implant 102L would initiate capture of the buffers 152L at a second time point (T2). That is, in conventional arrangements, the cochlear implant 102L would capture buffer 152L-(1) at a time delay/offset 154 relative to the capture of corresponding buffer 152R-(1). The presence of the time offset 154 means that, in conventional arrangements, the buffers 152L and 152R could include audio samples associated with non-contemporaneous audio content. For instance, with a time offset, a short audio signal (e.g., a click) could appear in buffer 152R-(1) of cochlear implant 102R, but appear in buffer 152L-(1) of cochlear implant 102L. Similarly, a longer audio signal might appear only in buffer 152R-(1) of cochlear implant 102R, but appear in both of buffer 152L-(1) and buffer 152L-(2) of cochlear implant 102L. Both situations would result in asynchronous or non-aligned stimulation data being presented at the cochlear implants 102R and 102L.

However, as shown in FIG. 2B, in accordance with embodiments presented herein, the cochlear implants 102L and 102R use the synchronized time base established via the binaural wireless link 162 to align the capture of audio samples for use in spectral analysis (e.g., ensure that contemporaneous audio content is being spectrally analyzed at both cochlear implants 102L and 102R at precisely the same time). For example, each of the cochlear implant 102L and cochlear implant 102R will determine that a synchronization of the binaural wireless link 162 has occurred and will have knowledge of the precise timing of that synchronization. In accordance with embodiments presented herein, the cochlear implant 102L and cochlear implant 102R are each configured to use this synchronization of the binaural wireless link 162, specifically the synchronization timing information, to synchronize (align) the spectral analysis at the cochlear implants 102L and 102R. For example, each of the cochlear implants 102L and 102R can start (including restart) the capture of buffers 152L and 152R at the same time, where the buffering start time is based on the synchronization timing of the wireless link 162. Therefore, as used herein, binaural “alignment” or “synchronization” of the spectral analysis refers to alignment/synchronization of the point in time the hearing devices, such as cochlear implants 102L and 102R, collect a buffer of audio samples for a corresponding spectral analysis (e.g., a determination is made at the same time to capture contemporaneous audio content).

As noted, if the capture of buffers 152L and 152R is started at the same time, the buffers 152L and 152R, when filled, will include contemporaneous audio content. As a result, the spectral analysis performed by the cochlear implants 102L and 102R on corresponding filled buffers will also occur at the same time, and will include contemporaneous audio content. Stated differently, the output of the spectral analysis at each of the cochlear implants 102L and 102R will be aligned in both time and in terms of contemporaneous audio content.

The cochlear implant 102L and cochlear implant 102R can determine the synchronization timing of the binaural wireless link 162 in any of a number of different manners. As noted above, in certain embodiments, a synchronization event/notification is generated (e.g., at the wireless transceivers 120L and 120R) indicating the synchronization timing of the binaural wireless link 162, and the cochlear implants 102L and 102R. However, this technique is merely illustrative and other techniques are possible. In one form, the synchronization event/notification is generated when the binaural wireless link 162 is established and the synchronization event delivers a relative time stamp to each cochlear implant 102L and 102R.

In certain embodiments, the synchronization event indicates, or is used to determine, an operational time difference between the cochlear implants 102L and 102R. For example, in the arrangement of FIG. 2B, the synchronization event can be used to determine that the cochlear implant 102L lags cochlear implant 102R by a time period 156 (e.g., difference between T1 and T2). As such, in accordance with one example of the techniques presented herein, the operations of cochlear implant 102R are adjusted to move the buffer capture to be aligned with that of cochlear implant 102L. These adjustments include not only the time that the audio capture is initiated, but will also change the actual audio content samples that are captured. That is, using the synchronization event, the cochlear implant 102R can determine what specific time point the cochlear implant 102L will capture, for example, buffer 152L-(1). Accordingly, in the example of FIG. 2B, the cochlear implant 102R adjusts operation so that the capture of buffer 152R-(1) will begin at the same time as that of 152L-(1) (e.g., processing module 124R with a filter-bank that operates over a buffer of audio samples time-aligns itself so that the buffer of audio samples over which the spectral analysis is calculated is aligned to the same point in time on both cochlear implants). If this capture occurs at the same time and with sufficient accuracy, then buffer 152R-(1) will begin at the same time as that of buffer 152L-(1) and will include audio samples that are associated with contemporaneous audio content.

FIG. 2B illustrates an embodiment in which the signal path of cochlear implant 102R is delayed so as to match that of cochlear implant 102L. Alternatively, the signal path of both cochlear implants 102L and 102R could be adjusted by half (or other percentage) of the difference between them using an agreed negotiation scheme. The techniques presented herein can be implemented, in part, using a programmable delay line in each hearing device that can be programmed adjust/slide, in time, the spectral analysis (e.g., adjust the audio capture point for subsequent spectral analysis). As noted, these embodiments are merely illustrative and, in certain embodiments, each of the cochlear implants 102L and 102R can start (which includes a restart) spectral analysis at some time determined relative to the synchronization event. The time to start spectral analysis can be, for example, when the synchronization event is received, some predetermined time after the synchronization event is received, etc.

As noted, the above process aligns/synchronizes the capture of the buffers 152L and 152R at cochlear implants 102L and 102R. In the cochlear implant system 100, this could be of benefit for localization as each spectral analysis (filter-bank analysis) period leads to a selection of maxima (channels to stimulate), and as long as timing in the further parts of the signal path and the implant interface is maintained, these will be delivered to the cochlea at the same time, and represent the same portion of input audio on both cochlear implants 102L and 102R.

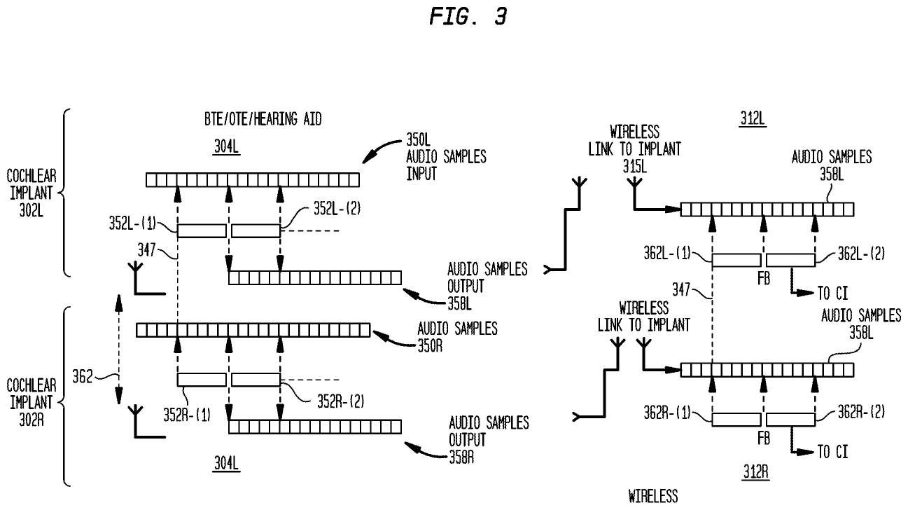

FIG. 2B illustrates an embodiment where the processing is performed at a single location on each side (e.g., at processing module 124L and 124R). As shown in FIG. 3, the techniques presented herein could be extended to a multi-lateral (four device hearing device system) having spectral analysis performed at two different locations on each side of the system (e.g., two spectral analyses on the left side and two spectral analyses on the right side).

More specifically, FIG. 3 illustrates an example cochlear implant system 300 comprising two cochlear implants, referred to as cochlear implant 302L and 302R. Cochlear implant 302L comprises an external component 304L and an implantable component 312L, while cochlear implant 302R comprises an external component 304R and an implantable component 312R. As shown in FIG. 3, each of the external components 304L and 304R and each of the implantable components 312L and 312R are configured to perform spectral analysis operations (e.g., there is filter-bank processing in the external device as well as the implantable devices).

In the arrangement of FIG. 3, if the time windows over which the different parts of the systems are calculated are not time aligned, there will be differences in the electrical stimulation presented in time. The techniques presented herein address this issue by extending the previously described system of FIG. 2 into two pairs of systems, where the first pair includes the external components 304L/304R and the second pair includes the implantable components 312L/312R. That is, the spectral analysis performed at each of the external components 304L and 304R are aligned with one another, as described above, while the spectral analysis performed at each of the implantable components 312L and 312R are aligned with one another. In certain embodiments, all four components are synchronized together, while in other embodiments there may be an offset between the external components and the implantable components. In the embodiment of FIG. 3, the transcutaneous link between the external components 304L/304R and the corresponding implantable component 312L/312 includes audio data.

More specifically, in the example of FIG. 3, the cochlear implants 302L and 302R communicate using a binaural wireless link 362, which is used to establish a synchronized spectral analysis time base, as described above with reference to FIG. 2B. As noted, in one specific example, the synchronized spectral analysis time base can be achieved using a “synchronization event” 347 when the binaural wireless link 362 is first established and/or could be a regular event in order to maintain synchronization.

In accordance with embodiments presented herein, the synchronized spectral analysis time base is used at the external component 304L (e.g., by spectral analysis synchronization logic) and the external component 304R (e.g., by spectral analysis synchronization logic) for binaural synchronization of the spectral analysis operations performed at the external components. More specifically, as shown in FIG. 3, each of the external components 304L and 304R receive a plurality of audio samples (e.g., via the respective sound input elements). The audio samples received by the external component 304L are referred to as audio samples 350L, while the audio samples received by the external component 304R are referred to as audio samples 350R.

As noted above, spectral analysis refers to a process to determine the frequency contents of received time domain sound signals. The spectral analysis is performed on a subset of the received audio samples collected, for example, in a buffer. To ensure binaural alignment of the spectral analysis, the synchronized spectral analysis time base is used to synchronize the capture of buffers at each of the external components 304L and 304R. For example, external component 304L collects different buffers 352L-(1)-352L-(N) of the audio samples 350L for spectral analysis, while external component 304L collects different buffers 352R-(1)-352R-(N) of the audio samples 350R for spectral analysis.

In the embodiment of FIG. 3, the external components 304L and 304R use the synchronized spectral analysis time base established via the binaural wireless link 362 to align the capture of audio samples for use in spectral analysis. That is, using the synchronized spectral analysis time base, the external components 304L and 304R can determine what specific time the other device will capture the buffers. Accordingly, the external component 304L and/or the external component 304R adjusts operation so that the capture of buffer 352R-(1) will begin at the same time as that of buffer 352L-(1). If this capture occurs at the same time and with sufficient accuracy, then the spectral analysis of buffer 352R-(1) will begin at the same time as the spectral analysis of buffer 352L-(1) and will include audio samples that are associated with contemporaneous audio content.

In the example of FIG. 3, the external component 304L and the external component 304R each perform the spectral analysis and, in certain embodiments, further processing operations to output a corresponding set of audio samples. These audio samples are then sent to the respective implantable component 312L and 312R. In particular, external component 304L outputs audio samples 358L that are generated from the audio samples 350L. The audio samples 358L are then sent, via a wireless link 315L (e.g., closely-coupled link, magnetic induction link, Bluetooth link, etc.), to the implantable component 312L. The audio samples 358L are generated from the audio samples 350L. Similarly, external component 304R outputs audio samples 358R that are generated from the audio samples 350R. The audio samples 358R are then sent, via a wireless link 315R, to the implantable component 312R.

In the embodiment of FIG. 3, the implantable components 312L and 312R also use the synchronized spectral analysis time base established from the binaural wireless link 362 to align the capture of audio samples 358L and 358R for use in spectral analysis.

As noted above, spectral analysis refers to a process to determine the frequency contents of received time domain sound signals. The spectral analysis is performed on a subset of the received audio samples collected, for example, in a buffer. To ensure binaural alignment of the spectral analysis, the synchronized spectral analysis time base is also used to synchronize the capture buffers at each of the implantable components 312L and 312R. For example, implantable component 312L collects different buffers 362L-(1)-362L-(N) of the audio samples 358L for spectral analysis, while implantable component 312R collects different buffers 362R-(1)-362R-(N) of the audio samples 358R for spectral analysis.

In certain embodiments, the synchronized time base indicates, or is used to determine, that the implantable component 312L lags implantable component 312R. As such, in this example, the operations of implantable component 312L and/or implantable component 312R are adjusted to align the buffer capture at each device. These adjustments include not only the time that the audio capture is initiated, but also the actual audio content samples that are captured. That is, using the synchronized time base, the implantable components 312L and 312R can determine what specific time the other device will capture the buffers. Accordingly, the implantable component 312L and/or the implantable component 312R adjusts operation so that the capture of buffer 362R-(1) will begin at the same time as that of buffer 362L-(1). If this capture occurs at the same time and with sufficient accuracy, then buffer 362R-(1) will begin at the same time as that of buffer 362L-(1) and will include audio samples that are associated with contemporaneous audio content.

As noted, the above process aligns/synchronizes the capture of the buffers 352L and 352R, as well as 362L and 362L, at the cochlear implants 302L and 302R. In the cochlear implant system 300, this could be of benefit for localization as each spectral analysis (filter-bank analysis) period leads to a selection of maxima (channels to stimulate), and as long as timing in the further parts of the signal path and the implant interface is maintained, these will be delivered to the cochlea at the same time, and will represent contemporaneous portions of input audio on both cochlear implants 302L and 302R.

As noted, FIG. 2B illustrates an embodiment in which the spectral analysis is aligned/synchronized at external components, while FIG. 3 illustrates an embodiment in which the spectral analysis is aligned/synchronized at external components and at implantable components. In another embodiment, the external components may not perform any spectral analysis, but rather only time domain audio sample processing. In these embodiments, the audio is then sent to the implantable components and the implantable components are the only parts of the system that perform spectral analysis. In such embodiments, the implantable components can operate as described above with reference to FIG. 2B or 3 to align/synchronize their spectral analysis operations with one another. Another similar example is when the implantable components are sent an audio stream from any type of external accessory, Bluetooth stream or phone, etc. In such embodiments, the bilateral wireless link is still used to synchronize spectral analysis at the implantable components, without any equivalent operation on the external components.

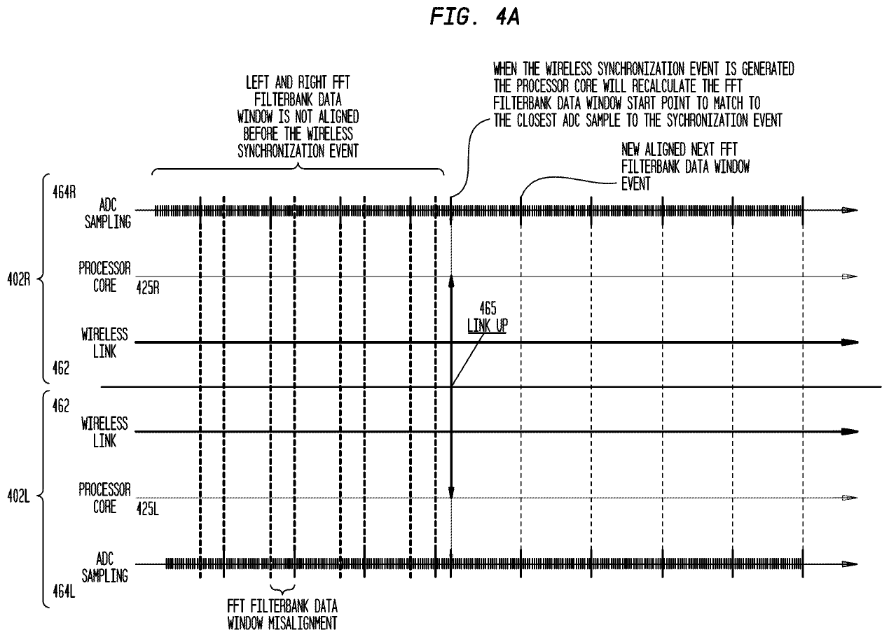

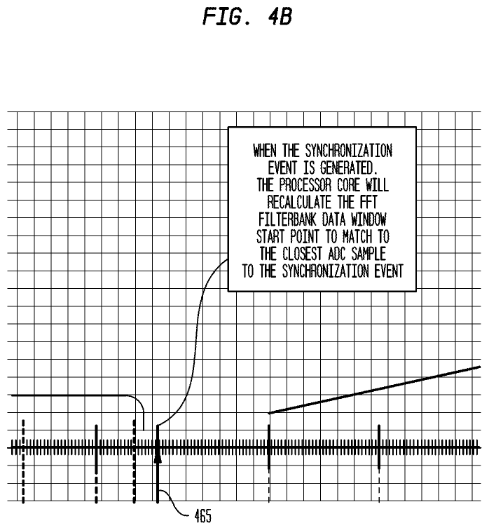

In accordance with certain examples presented herein, the buffers are generally “data windows” or “data buffers” for use in a spectral analysis process, such as a fast Fourier transform (FFT) or similar filter-bank. FIG. 4A illustrates one such example for a (left/right) pair of hearing devices 402L and 402R including processing cores 425L and 425R, respectively.

More specifically, FIG. 4A illustrates that, initially, the hearing devices 402L and 402R are “free running,” and neither the A/D audio sampling (A/D audio sample periods) 464L/464R nor the “data windows” of audio samples over which the FFT filter-banks are calculated are aligned in time (i.e., there is FFT filter-bank data misalignment), which is the case with conventional arrangements. However, in accordance with embodiments presented herein, characteristics of a binaural wireless link 462 is used for synchronization/alignment of the data window capture. For example, as part of the wireless negotiation of the wireless link 462 between the hearing devices 402L and 402R, timing slots are determined by, for example, a wireless protocol. When those timing slots are defined, each hearing device 402L and 402R knows precisely when the other device can send or receive data. At the moment this negotiation is complete, both hearing devices 402L and 402R can issue a synchronization event (or similar) indication/notification 465 that the wireless link 462 has been established. This event will happen simultaneously on each of the hearing devices 402L and 402R or at a known offset on each side or to the event (if there an offset). Assuming, for example, that the synchronization event 465 is simultaneous on both hearing devices 402L and 402R, then FIG. 4A shows how the event 465 can be used to “align” the data window over which the FFT filter-bank calculation is performed. If there is an offset, then this can also be taken into account in the calculation of the new data window. If continuous or occasional realignment is needed, then the synchronization event 465 might be called “realignment,” and the process just described could also happen regularly.

In the example of FIG. 4A, the wireless synchronization event 465 is generated at a time ‘T.’ Using the synchronized time base, as determined from the wireless synchronization event 465, the processor cores 425L and 425R at the hearing devices 402L and 402R, respectively, will re-calculate the FFT filter-bank data window start point to match the closest ADC sample to the synchronization event 465. That is, the buffer captures (FFT filter-bank data window) are aligned across the hearing devices 402L and 402R to start at a closest audio sample (A/D sample).

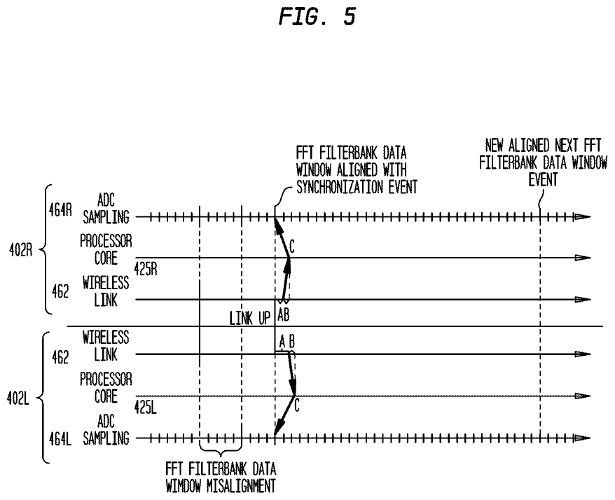

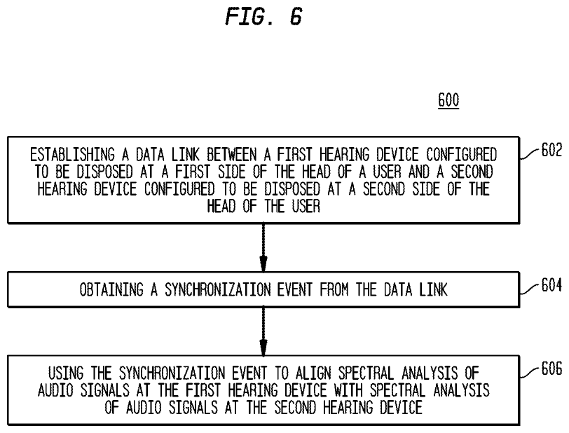

In general, if the buffer (FFT data window) alignment is very different across the hearing devices 402L and 402L before the synchronization or realignment event, then a portion of audio samples can be discarded (leaving a small gap in the sound) or a portion of the previous data can be overwritten with a new calculation. Alternatively, a smoothing operation or interpolation operation can be used between alignment events, if there is a need to avoid any discontinuities in the sound. However, given that FFT calculations are often overlapped, then any shift in the overlap point (due to the synchronization event) is likely to be minor in terms of effects on the sound presented to the cochlear implant recipient. FIG. 5 illustrates further details of the wireless synchronization event with delays, again with reference to the same arrangement as in FIGS. 4A and 4B.

More specifically, in practice, the wireless synchronization event will be communicated to the processor cores 425L and 425R with some delay (assuming that there is a wireless chip/sub-system separate from the sound processor core(s)). One solution is that a time counter can be started on the wireless sub-system when the synchronization event occurs. An amount of time, indicated by ‘A’ in FIG. 5, will pass before the link event is communicated to the processor core. The event that is generated to the processor core will carry the exact value of this synchronization time counter that represents the period indicated by ‘A’. The transfer of the event from the wireless sub-system (which can be on the same chip as the processor core or on another chip than the processor core) to the processor core will have a fixed known delay indicated in FIG. 5 by ‘B’. With the knowledge of time period ‘A’ and ‘B,’ the processor core, at time moment ‘C,’ combines this information with the knowledge of the ADC sampling subsystem to calculate the exact ADC sample at which the wireless synchronization event was generated. By executing this process on the left and right hearing device it is possible to synchronize/align the spectral analysis between both sides. This can also occur at regular intervals to maintain alignment.

FIG. 6 is a flowchart of an example method 600, in accordance with certain embodiments presented herein. Method 600 begins at 602 where a binaural wireless data link is established between a first hearing device configured to be worn on a first side of the head of a user and a second hearing device configured to be worn on a second side of the head of the user. At 604, a synchronization event is obtained from establishment of the binaural wireless data link. At 606, the synchronization event is used to align spectral analysis of audio signals at the first hearing device with spectral analysis of audio signals at the second hearing device

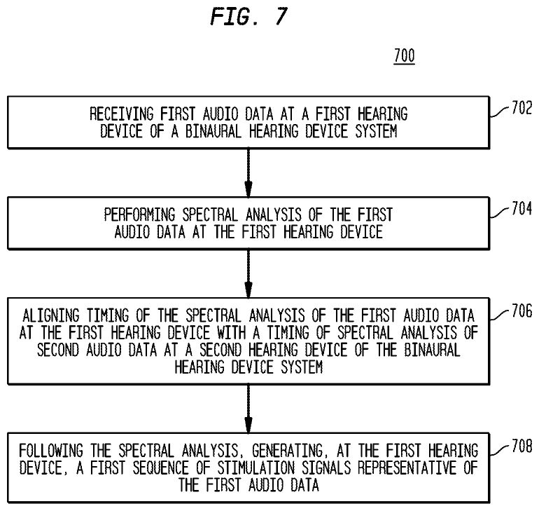

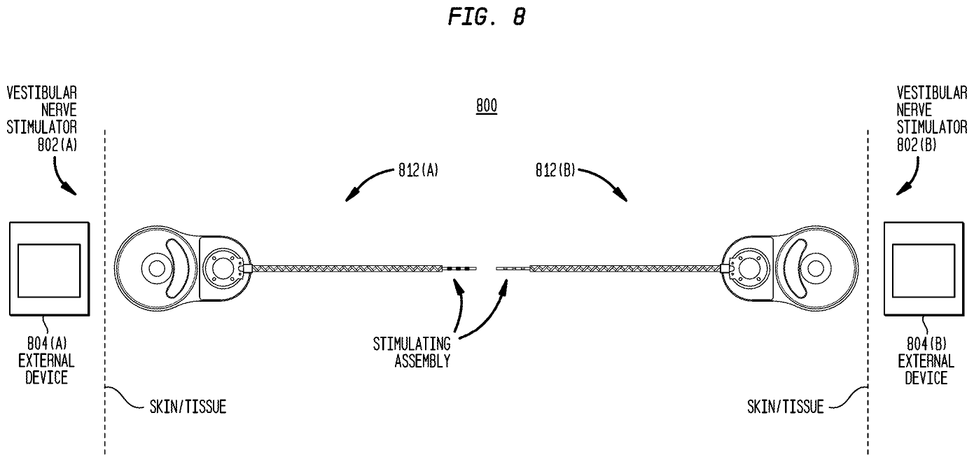

FIG. 7 is a flowchart of a method 700 in accordance with certain embodiments presented herein. Method 700 begins at 702 where a first hearing device of a hearing device system receives first audio data. At 704, the first hearing device performs spectral analysis of the first audio data. At 706, following the spectral analysis, the first hearing device generates a first sequence of stimulation signals representative of the first audio data. At 708, the first hearing device aligns timing of the spectral analysis of the first audio data at the first hearing device with a timing of spectral analysis of second audio data at a second hearing device of the hearing device system.

Merely for case of description, the techniques presented herein have primarily described herein with reference to an illustrative medical device system, namely a cochlear implant system that delivers electrical stimulation to both ears of a recipient. However, it is to be appreciated that the techniques presented herein may also be used with a variety of other medical devices that, while providing a wide range of therapeutic benefits to recipients, patients, or other users, may benefit from the techniques presented. For example, a cochlear implant system in accordance with embodiments presented herein may also deliver acoustic stimulation to one or both ears of the recipient (e.g., one or more of the cochlear implants is an electro-acoustic cochlear implant). It is also to be appreciated that the two cochlear implants of a cochlear implant system in accordance with embodiments presented need not be identical with respect to, for example, the number of electrodes used to electrically stimulate the cochlea, the type of stimulation delivered, etc.

Furthermore, it is to be appreciated that the techniques presented herein may be used with other systems including two or more devices, such as systems including one or more personal sound amplification products (PSAPs), one or more acoustic hearing aids, one or more bone conduction devices, one or more middle ear auditory prostheses, one or more direct acoustic stimulators, one or more other electrically simulating auditory prostheses (e.g., auditory brain stimulators), one or more vestibular devices (e.g., vestibular implants), one or more visual devices (i.e., bionic eyes), one or more sensors, one or more pacemakers, one or more drug delivery systems, one or more defibrillators, one or more functional electrical stimulation devices, one or more catheters, one or more seizure devices (e.g., devices for monitoring and/or treating epileptic events), one or more sleep apnea devices, one or more electroporation devices, one or more remote microphone devices, one or more consumer electronic devices, etc. For example, FIGS. 8, 9, and 10 are schematic diagrams of alternative systems that can implement aspects of the techniques presented herein.

More specifically, FIG. 8 is a schematic diagram illustrating an example vestibular system 800 that can be configured to perform synchronized spectral analysis, in accordance with certain embodiments presented herein. In this example, the vestibular system 800 comprises a first vestibular stimulator 802(A) and a second vestibular stimulator 802(B). The first vestibular stimulator 802(A) comprises an external device 804(A) and an implantable component 812(A), while the second vestibular stimulator 802(B) comprises an external device 804(B) and an implantable component 812(B). In accordance with certain embodiments presented herein, the first vestibular stimulator 802(A) (e.g., external device 804(A) and/or implantable component 812(A)) and/or the second vestibular stimulator 802(B) (e.g., external device 804(B) and/or implantable component 812(B)) are configured to implement aspects of the techniques presented herein to perform synchronized spectral analysis of received/input signals (e.g., audio signals, sensor signals, etc.).

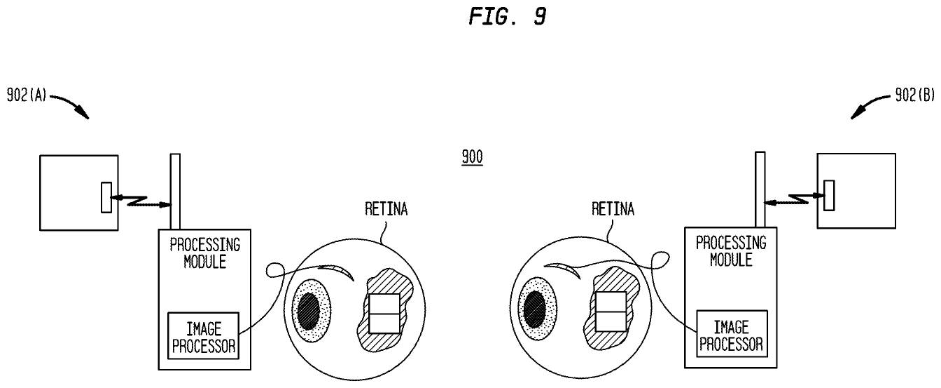

FIG. 9 is a schematic diagram illustrating an example retinal prosthesis system 900 that can be configured to perform synchronized spectral analysis, in accordance with certain embodiments presented herein. In this example, the retinal prosthesis system 900 comprises a first retinal prosthesis 902(A) and a second retinal prosthesis 902(B). In accordance with certain embodiments presented herein, the first retinal prosthesis 902(A) and/or the second retinal prosthesis 902(B) are configured to implement aspects of the techniques presented herein to perform synchronized spectral analysis of received/input signals (e.g., light signals, sensor signals, etc.).

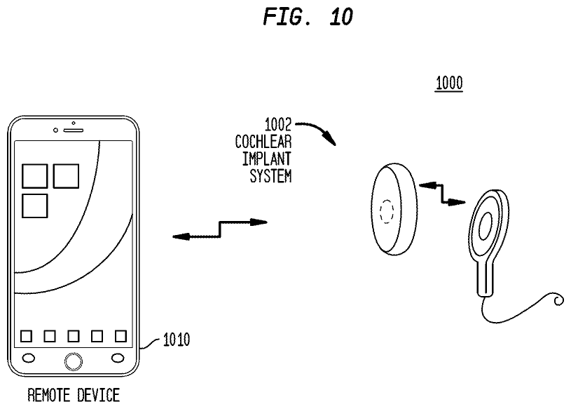

FIG. 10 is a schematic diagram illustrating another example system 1000 that can be configured to perform synchronized spectral analysis, in accordance with certain embodiments presented herein. In this example, the system 1000 comprises an external device 1010 (e.g., mobile phone) and a cochlear implant system 1002. In accordance with certain embodiments presented herein, the external device 1010 and/or the cochlear implant system 1002 are configured to implement aspects of the techniques presented herein to perform synchronized spectral analysis of received/input signals (e.g., audio signals, sensor signals, etc.).

As previously described, the technology disclosed herein can be applied in any of a variety of circumstances and with a variety of different devices. While the above-noted disclosure has been described with reference to medical device, the technology disclosed herein may be applied to other electronic devices that are not medical devices. For example, this technology may be applied to, e.g., ankle or wrist bracelets connected to a home detention electronic monitoring system, or any other chargeable electronic device worn by a user.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
establishing a data link between a first device configured to be disposed at a first side of a head of a user and a second device configured to be disposed at a second side of the head of the user;
obtaining a synchronization event from the data link; and
using the synchronization event to align spectral analysis of audio signals at the first device with spectral analysis of audio signals at the second device.

2. The method of claim 1, wherein using the synchronization event to align spectral analysis of audio signals at the first device with spectral analysis of audio signals at the second device includes:
determining a relative operational time difference between the first device and the second device from the synchronization event.

3. The method of claim 1, wherein using the synchronization event to align spectral analysis at the first device with spectral analysis of second device comprises:
obtaining a first set of audio samples at the first device; and
capturing a buffer of the first set of audio samples at the first device,
wherein a timing of the capture of the buffer of the first set of audio samples is determined based on the synchronization event.

4. The method of claim 3, wherein a timing of the capture of the buffer of the first set of audio samples is determined based on synchronization event comprises:
determining a time offset from the synchronization event to start capture of the buffer of the first set of audio samples.

5. The method of claim 3, further comprising:
applying a fast Fourier transform (FFT) filter-bank to the buffer of the first set of audio samples at the first device.

6. The method of claim 3, further comprising:
generating, at the first device, stimulation signals from the buffer of the first set of audio samples.

7. The method of claim 6, wherein the first device is a cochlear implant, and wherein generating the stimulation signals from the buffer of the first set of audio samples comprises:
generating electrical stimulation signals from the buffer of the first set of audio samples.

8. The method of claim 3, further comprising:
obtaining a second set of audio samples at the second device; and
capturing a buffer of the second set of audio samples at the second device,
wherein a timing of the capture of the buffer of the second set of audio samples is determined based on the synchronization event and is aligned in time with the capture of the buffer of the first set of audio samples at the first device.

9. The method of claim 8, wherein the second device is a cochlear implant, and wherein the method further comprises:
generating, at the second device, electrical stimulation signals from the buffer of the second set of audio samples.

10. The method of claim 1, wherein the first device comprises a first external component and a first implantable component each configured to perform spectral analysis, and wherein using the synchronization event to align spectral analysis of audio signals at the first device with spectral analysis of audio signals at the second device comprises at least one of:
synchronizing spectral analysis performed at the first external component with spectral analysis performed at a second external component of the second device, or
synchronizing spectral analysis performed at the first implantable component with spectral analysis performed at a second implantable component of the second device.

11. The method of claim 1, further comprising:
receiving first audio data at the first device;
performing spectral analysis of the first audio data at the first device;
using the synchronization event to align a timing of the spectral analysis of the first audio data at the first device with a timing of spectral analysis of second audio data at the second device; and
following the spectral analysis, generating, at the first device, a first sequence of stimulation signals representative of the first audio data.

12. The method of claim 11, further comprising:
converting the first audio data into a first set of audio samples; and
capturing a subset of the first set of audio samples at the first device.

13. The method of claim 11, wherein aligning timing of the spectral analysis of the first audio data at the first device with the timing of the spectral analysis of second audio data at the second device comprises:
aligning a start of the capturing of the subset of the first set of audio samples at the first device with a capturing of a subset of second set of audio samples at the second device such that the subset of the first set of audio samples and the subset of the second set of audio samples include contemporaneous audio content.

14. The method of claim 13, wherein aligning a start of the capture of the subset of the first set of audio samples at the first device with a capture of the subset of the second set of audio samples at the second device comprises:
delaying a start of the capturing of the subset of the first set of audio samples at the first device to correspond to a start of the capturing of the subset of the second set of audio samples at the second device.

15. The method of claim 13, wherein aligning a start of the capture of the subset of the first set of audio samples at the first device with the capture of the subset of the second set of audio samples at the second device:
aligning the start of the capture of the subset of the first set of audio samples at the first device with a capture of the subset of the second set of audio samples at the second device to a closest audio sample.

16. The method of claim 11, further comprising:
determining a relative operational time difference between the first device and the second device; and
using the relative operational time difference to align the spectral analysis of the first audio data at the first device with the timing of the spectral analysis of the second audio data at the second device.

17. The method of claim 16, wherein determining the relative operational time difference between the first device and the second device comprises:
determining the relative operational time difference based on at least one characteristic of a wireless data link established between the first device and the second device.

18. The method of claim 17, wherein determining the relative operational time difference based on at least one characteristic of the wireless data link comprises:
determining the relative operational time difference from a synchronization event generated from the wireless data link.

19. (canceled)

20. A system, comprising:
a first device located at a first ear of a user and including one or more first processors configured to:
obtain a first set of audio samples, and
capture one or more buffers of the first set of audio samples; and
a second device located at a second ear of the user and including one or more second processors configured to:
obtain a second set of audio samples, and
capture one or more buffers of the second set of audio samples,
wherein the one or more first processors and the one or more second processors are configured to cooperate to synchronize the capture of the one or more buffers of the first set of audio samples with the capture of the one or more buffers of the second set of audio samples.

21. The system of claim 20, wherein the one or more first processors are configured to perform spectral analysis of the one or more buffers of the first set of audio samples to generate first frequency domain signals, and wherein the one or more second processors are configured to perform spectral analysis of the one or more buffers of the second set of audio samples to generate second frequency domain signals.

22. The system of claim 21, wherein the one or more first processors are configured to perform sound processing operations on the first frequency domain signals, and wherein the one or more second processors are configured to perform sound processing operations on the second frequency domain signals.

23. The system of claim 20, wherein to synchronize the capture of the one or more buffers of the first set of audio samples with the capture of the one or more buffers of the second set of audio samples, the one or more first processors or the one or more second processors are configured to:
align a start of the capture of the one or more buffers of the first set of audio samples at the first device with a capture of the one or more buffers of the second set of audio samples at the second device so that the one or more buffers of the first set of audio samples and the one or more buffers of the second set of audio samples include contemporaneous audio content.

24. The system of claim 23, wherein to align a start of the capture of the one or more buffers of the first set of audio samples at the first device with the capture of the one or more buffers of the second set of audio samples, the one or more first processors are configured to:
delay a start of the capturing of the one or more buffers of the first set of audio samples at the first device to correspond to a start of the capturing of the one or more buffers of the second set of audio samples at the second device.

25. The system of claim 23, wherein to align a start of the capture of the one or more buffers of the first set of audio samples at the first device with the capture of the one or more buffers of the second set of audio samples, the one or more second processors are further configured to:
advance a start of the capturing of the one or more buffers of the second set of audio samples at the second device.

26. The system of claim 23, wherein the one or more first processors or the one or more second processors are configured to:
determine a relative operational time difference between the first device and the second device; and
using the relative operational time difference to synchronize the capture of the one or more buffers of the first set of audio samples with the capture of the one or more buffers of the second set of audio samples.

27. The system of claim 26, wherein the one or more first processors or the one or more second processors are configured to determine the relative operational time difference between the first device and the second device based on at least one characteristic of a data link established between the first device and the second device.

28. The system of claim 27, wherein the one or more first processors or the one or more second processors are configured to determine the relative operational time difference from a synchronization event generated from the data link.

29. (canceled)

30. Non-transitory computer readable storage media encoded with instructions that, when executed by one or more processors of a first device configured to be located at a first side of the head of a user, cause the one or more processors to:
establish a data link between the first device and a second device configured to be located at a second side of the head of the user;
obtain a synchronization event from the data link; and
use the synchronization event to align spectral analysis of audio signals at the first device with spectral analysis of audio signals at the second device.

31. The non-transitory computer readable storage media of claim 30, wherein the instructions to use the synchronization event to align spectral analysis of audio signals at the first device with spectral analysis of audio signals at the second device comprise instructions that, when executed by the one or more processors, cause the one or more processors to:
determine a relative operational time difference between the first device and the second device from the synchronization event.

32. The non-transitory computer readable storage media of claim 31, wherein the instructions to use the synchronization event to align spectral analysis of audio signals at the first device with spectral analysis of audio signals at the second device comprise instructions that, when executed by the one or more processors, cause the one or more processors to:
obtain a first set of audio samples at the first device; and
capture a subset of the first set of audio samples at the first device,
wherein a timing of the capture of the subset of the first set of audio samples is determined based on the synchronization event.

33-49. (canceled)
